(19) United States
(12) Patent Application Publication
(10) Pub. No.: US 2024/0426803 A1
(43) Pub. Date: Dec. 26, 2024

(54) METHOD FOR PREPARATION, DETECTION, AND ANALYSIS OF SYNTHETIC POLYMERS USING AUTOMATED MINERALOGY SYSTEMS

(71) Applicant: CGG SERVICES SAS, Massy Cedex (FR)

(72) Inventors: Gareth ROGERS, Bangor (GB); Amy MCGARRY, Conwy (GB)

(21) Appl. No.: 18/694,143

(22) PCT Filed: Oct. 14, 2021
(86) PCT No.: PCT/IB2021/000711

§ 371 Date: Mar. 21, 2024

(51) Int. Cl. G01N 33/24; G01N 1/28; G01N 1/36

(52) U.S. Cl. CPC G01N 33/24; G01N 1/286; G01N 1/36; G01N 2001/366

(57) ABSTRACT

A method for preparing a block for chemical analysis includes providing a substantially inorganic mounting medium; providing a sample material that includes plastic and another material; mixing the substantially inorganic mounting medium with the sample material to generate the block; and smoothing a first surface of the block to expose the plastic.

### BACKGROUND

### Technical Field

Embodiments of the subject matter disclosed herein relate to methods for preparing and analyzing a probe for synthetic polymer detection, and more specifically, to a methodology for mixing a sample material with a mounting medium to form a substrate that is suitable for existing automated mineralogic systems, and also for using the existing software of the automated mineralogic systems for detecting the synthetic polymers in the substrate, and identifying the type and quantity of the synthetic polymer in the substrate.

### Discussion of the Background

Plastic is used today in every sector of life. Due to the advanced manufacturing processes, the generated plastic has not only improved its characteristics in terms of strength, appearance, reduced weight, but also its cost went down, making it one of the most used materials both in the residential and industrial environment. Plastic is fundamentally a synthetic polymer that is non-biodegradable in nature. Due to the existing large amount of plastic, the lack of enough recycling facilities because the low profit derived from such activities, and also due to human behavior, large amounts of the used plastic find their ways in the environment, both on land and in the oceans. Because the plastic remains stable for thousands of years without any degradation, the impact of this waste plastic on the environment is very damaging.

Various projects and techniques are proposed for removing the waste plastic from the environment and recycling it in an efficient manner. However, to be able to efficiently remove the waste plastic from the environment, there is a need for a method for identifying the type of plastic and the amount of plastic that is present in a given sample, i.e., soil sample. While the large pieces of plastic are visible with the bare eye in the environment (called herein “macroplastic”), there is also a large amount of tiny particles (microscopic) of plastic (called herein “microplastic”) in the environment, which are not necessarily visible as their size is very small, and these microplastics are the unseen threat to the environment, which also needs to be removed and recycled. To deal with the microplastics, a method for identifying the type of plastic and the amount of plastic should be capable of doing this analysis on the microscopic plastics.

To date, to analyze synthetic polymers, including macro-to microplastic particles within a substrate, it is generally necessary to isolate the plastic using complex procedures. However, the existing scanning electron microscope (SEM)-based automated minerology techniques enable particles of interest to be examined without necessitating prior separation from the surrounding material. Automated minerology techniques include: QEMSCAN/QEMSCAN Wellsite (Quantitative Evaluation of Materials by Scanning Electron Microscopy); MLA (Mineral Liberation Analyzer), Mineralogic, INCAMineral, TIMA (Tescan integrated mineral analyzer), AMICS (Automated Rapid Scanning for Mineral and Rock Characterization by SEM), RoqSCAN (a suite of software procedures for identifying minerals and hydrocarbons developed by CGG, the assignee of this application), and MinSCAN.

Standard preparation methods for automated mineralogy analyses generally involve the setting of the sample material 102, as shown in FIG. 1A, into an epoxy resin 108, which forms the mounting medium, as reported in the relevant scientific literature. The sample material 102 may include, in addition to the mineral of interest 104, the surrounding substrate 106 of the mineral 104. The epoxy resin 108 both binds and penetrates the sample material 102, filling regions of empty space. The obtained block 100 is then ground and polished to produce a smooth, even and flat surface 110 for analysis, as shown in FIG. 1B. Often, a thin layer of carbon or a metal 112, in the order of a few tens of nm thickness, is applied to the surface 110 of the block 100 (see FIG. 1B). This is necessary especially for samples that are beam sensitive, such as biological substances and polymers, which would otherwise get damaged by the high-energy electron beam of the analyzing device, and/or samples that are non-conductive, which would result in charge-build-up. Carbon is typically used as a coating (layer 112) because its X-ray peaks do not conflict with the peaks of other elements (minerals), thus facilitating analysis.

The traditional automated mineralogical analysis (i.e., the software commands that are run on the analyzing system, for example, QEMSCAN) involves two types of measurement: (1) collection of energy dispersive x-ray (EDS) data to measure different elemental concentrations, and (2) collection of backscattered electrons (BSE) data to distinguish different materials based on variations in density. Most of the time, these two measurements are taking place simultaneously, with different sensors.

The epoxy resin 108 has a significantly lower density than the typical minerals 104 and rock 106, which are often analyzed by automated mineralogy analysis systems. In this regard, note that the typical preparation methods and associated systems scan for the presence of Au or Cu or other minerals in the sample that have a high density. For example, in the standard QEMSCAN process, the porosity of a material is determined based on a sub-25 RGB (the RGB color model relies on red, green, and blue colors, with the lowest color intensity being 0 and the highest color intensity being 255) BSE density calculation. This means that the system is set up to identify regions below 25 RGB (the epoxy region has the density corresponding to 25 RGB or lower) but then ignore such regions (by not recording corresponding EDS and BDS measurements), such that during the analysis process, the SEM is programmed to identify regions occupied by materials that exceed the density of the epoxy resin as only those minerals are of interest, and not the epoxy resin, and the regions corresponding to the epoxy region is classified as “background.” Not that it is not possible to quantify the background in the resulting dataset unless the point is inside the boundary of a rock particle, in which case it can be quantified as a porosity. The analysis occurs at micro-second speed, i.e., a point is rapidly measured to see if RGB is below 25. If below 25, the beam moves to next point. If above 25, the RGB value is recorded and EDS measurements are recorded before moving to the next point. This process repeats at a rate of around 200-300 times per second (i.e., ˜200-300 points evaluated per second).

Therefore, data is collected on the rocks/minerals of interest and not on the lower density material and void space (i.e., space between particles or pore space within particles), thus maximizing the time spent analyzing the material of interest. Once the process is complete, the data from the X-Y coordinates of each measurement are referenced to the mineral dictionary classification, enabling the construction of a mineral map image displaying the mineral and textural data of the rock/minerals, as shown in FIG. 2. By ignoring any material that has a density below 25 RGB, the traditional systems and algorithms are faster. However, this configuration of the existing mineral analyzers would miss detecting the synthetic polymers as they have a density similar to the density of the epoxy material.

Thus, there is a need for a new method that is capable of determining the location of the macro- and/or microplastics in a given sample while using the traditional analyzing systems.

### BRIEF SUMMARY OF THE INVENTION

According to an embodiment, there is a method for preparing a block for chemical analysis, and the method includes providing a substantially inorganic mounting medium, providing a sample material that includes plastic and another material, mixing the substantially inorganic mounting medium with the sample material to generate the block, and smoothing a first surface of the block to expose the plastic.

According to another embodiment, there is a block for chemical analysis in an automatic mineralogic analysis system. The block includes a substantially inorganic mounting medium, a sample material that includes plastic and another material, a first face of the block includes a part of each of the substantially inorganic mounting material, the plastic and the another material, and a second surface, opposite to the first surface and including only the substantially inorganic mounting medium.

According to yet another embodiment, there is a method for analyzing a probe with a mineralogic analyzing system, and the method includes placing the probe in the mineralogic analyzing system, setting a backscattered electron collection brightness at substantially zero to capture carbon-based materials, setting a spectral count rate between 100 and 5000 counts per pixel spectrum for capturing the carbon-based materials, and performing an analysis of the probe to identify a plastic present in the probe. The probe includes the plastic and a substantially inorganic mounting medium.

According to yet another embodiment, there is a non-transitory computer readable medium including computer executable instructions, wherein the instructions, when executed by a processor, implement the methods discussed above.

### BRIEF DESCRIPTION OF THE DRAWINGS

For a more complete understanding of the present invention, reference is now made to the following descriptions taken in conjunction with the accompanying drawings, in which:

FIG. 1A is a top illustration of a block to be analyzed in a mineralogic analyzing system, and the block includes a mineral of interest encased in a carbon epoxy medium, while FIG. 1B is a cross-section through the block;

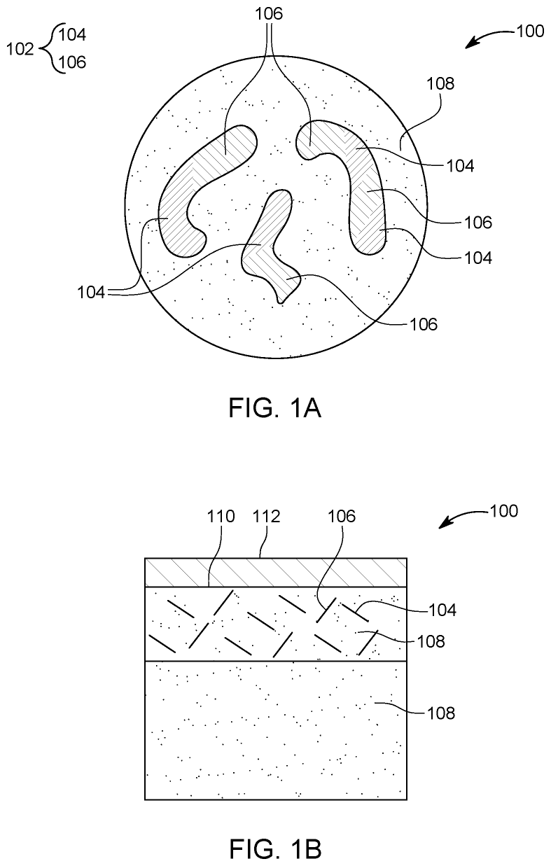

FIG. 2 illustrates a mineralogic map obtained for the block above with the mineralogic analyzing system;

FIG. 3 is a flow chart of a method for preparing a novel block that does not use the carbon epoxy medium;

FIG. 4 illustrates sample blocks using an inorganic mounting medium, which is used to encase plastic;

FIG. 5 is a flow chart of a method for preparing the block from the inorganic mounting medium and the plastic;

FIG. 6 is a cross-section through the novel block that uses the inorganic mounting medium;

FIG. 7 is a flow chart of a method for using the block with the inorganic mounting medium in a mineralogic analysis system and setting the system for detecting the plastic in the block;

FIG. 8 shows a plastic map obtained with the block having the inorganic mounting medium and using the new settings for plastic detection;

FIG. 9 is a flow chart of a method for forming the block, a method for creating a dictionary of materials to be compared to, and a method for analyzing the block and identifying the materials of the block based on the dictionary of the materials; and

FIG. 10 is a schematic of the mineralogic analyzing system that uses the block with the inorganic mounting medium and the new settings for plastic detection.

### DETAILED DESCRIPTION OF THE INVENTION

The following description of the embodiments refers to the accompanying drawings. The same reference numbers in different drawings identify the same or similar elements. The following detailed description does not limit the invention. Instead, the scope of the invention is defined by the appended claims.

Reference throughout the specification to “one embodiment” or “an embodiment” means that a particular feature, structure or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments.

According to an embodiment, a novel technique involves the application of an inorganic mounting medium to prepare samples for SEM analysis. The sample preparation method described herein is applicable to any methodology for analyzing plastic by SEM-based automated mineralogy techniques. According to another embodiment, which may be combined with the previous embodiment, the automated mineralogical analysis of samples includes the development of a Species Identification Protocol (SIP). This involves the collection of EDS spectral data on standard polymers and organic or non-plastic carbon-based or naturals materials, which may be found within the substrate, to build a database (which can alternatively be referred to as a “dictionary”). Particles observed in the acquired mineral map image of the sample can be compared against this database in order to confirm their identity. Once a SIP dictionary has been built with data from sufficient known samples of plastics and natural materials that may occur in a sediment sample, for example, pure standards, the dictionary then also becomes a self-referencing tool as more suspected and unknown plastics and natural materials are added to the dictionary. For example, when a sample is analyzed and review of the data suggests a particular particle is a likely plastic or natural, creating a SIP entry for this data point enables the software to automatically detect if it matches or is similar to known plastics and natural materials already in the dictionary. If this is the case, as a screening tool, these SIP entries can remain in the dictionary when analyzing a set of samples, and there is a tested and valid reason for concluding the material is a likely plastic or natural. In other words, as the dictionary grows, it becomes a self-referencing tool as more and more plastic and natural SIP entries are added, and also helps to clarify and quantify particles where it cannot be determined whether they are either plastic or natural. This in turns strengthens the plastic and natural categories, as anything surviving in these categories has been rigorously tested. This step of ‘self-referencing’ of the SIP is not found in the existing automated mineralogical analysis systems. This active step of ‘self-referencing’ using the SIP is being applied in a reverse of normal automated mineralogical analysis, where over 4,000 theoretical mineral elemental compositions are typically provided as standard with manufacturer systems. Users improve their SIPs by adding elemental data from pure standards alongside these theoretical and modified theoretical compositions. In this way, rock mineral compositions have a thorough theoretical dataset ready to compare any unknown rock minerals present within a sample. For the identification of plastics and natural materials, there are no theoretical elemental compositions provided by the manufacturer for different natural material and plastic samples, in particular since the method is predominantly reliant on the use of epoxy resin which would make the provision of this data irrelevant. Therefore, according to this embodiment, a self-referencing SIP is built from scratch, by obtaining a new dataset of elemental compositions of plastic and natural material responses in the SEM using pure standards. Then, in reverse, it is possible to build new theoretical and modified-theoretical dictionary entries once there is enough pure standards in the SIP for the software to begin to detect clashes.

The approach described here is applicable to any SEM-based automated minerology technique, and although software packages are system-specific, this methodology is consistent with all of them in as far as mineral dictionary design and SEM settings.

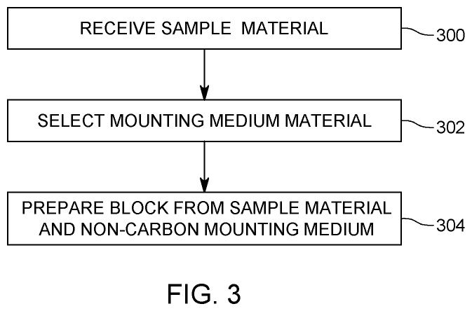

These embodiments are now discussed in more detail with regard to the figures. A sample material that includes macro- and/or microplastics and other chemical elements/materials is received in step 300, as illustrated in FIG. 3. The sample material may be directly collected from any part of the globe (soil from the garden, subsurface material, cuttings from a well, material from a mine, or even solid material recovered from a body of water), or may be received by the operator of the analyzing system from a client. The sample material needs to be mounted on a mounting medium prior to being analyzed by the analyzing system. Note that most of the known analyzing systems may be programmed, as discussed later, to handle this probe. However, for simplicity, this and other embodiments refer to QEMSCAN analyzing system.

The mounting medium is selected in step 302 to be an inorganic material. An “inorganic” material is defined herein as being at least one of (1) any inorganic substance, i.e., any substance that does not feature carbon-hydrogen bonds, and (2) an organometallic compound, i.e., a compound that includes a metal or metalloid directly bonded to a carbon atom. According to this definition, any hydrocarbon compound is an organic material, not an organometallic compound, and thus not an inorganic material. According to this definition, an inorganic substance that contain carbon includes one or more of silicon carbide, calcium carbonate, copper(I) acetylide, calcium cyanamide. Diamond and graphite are considered inorganic substances although these are both pure carbon materials (they contain no carbon-hydrogen bonds, they contain no hydrogen at all). Note that calcium acetate and sodium formate, for example, are considered to be inorganic substances in the art, although they do contain carbon, and also have C—H bonds. While the term “inorganic” material is defined above as not including carbon-hydrogen bonds, in one embodiment it is possible that a small amount of organic material is added to the inorganic mounting medium. For this case, i.e., inorganic material mixed with a small amount of organic material, the term “substantially inorganic material” is introduced and is defined as mainly containing an inorganic material, with the possibility of also including some organic material, but not more than 20% by mass of the total mounting medium. In one embodiment, the organic material is not more than 10% by mass of the total mounting medium. While the next embodiments are discussed with regard to an inorganic mounting medium, the same teachings apply to a “substantially inorganic material” mounting medium.

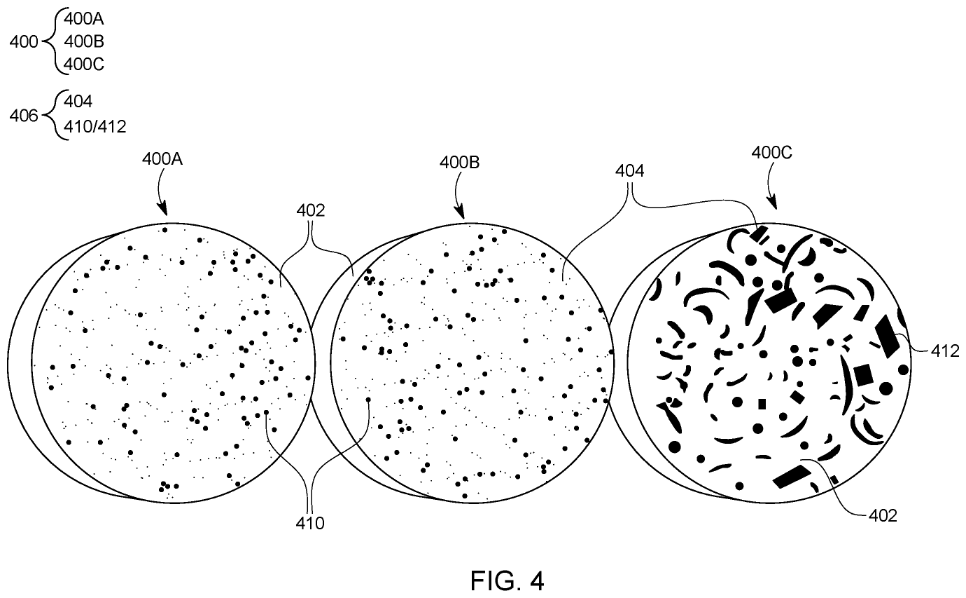

The mounting medium is selected based on its chemistry, to be able to identify plastics in the sample, i.e., to have a signature that is different from the signature of the plastic materials. In one embodiment, the mounting medium is selected to have a density greater than a density of the plastic material, if the density is considered to be signature of the material. FIG. 4 shows a probe 400 that may be implemented as three different probes 400A, 400B, and 400C. Probes 400A and 400B show microplastics 410 and other materials 404 (which are normally found in the soil) encased in a Crystacal mounting medium 402, while FIG. 400C shows macroplastics 410 and the other materials 404 encased by the Crystacal mounting medium 402. The other material 404 may be any material besides the plastic, for example, various components of the soil in which the plastic resides. The plastic 410/412 together with the other material 404 are called herein the sample material 406. Note that the other material may include one or more materials, which may be similar or not. For each probe, the mounting medium 402 is a substantially inorganic material, which a signature different from the signature (e.g., density) of the micro- and macro-plastics. The condition of having the signature of the mounting medium larger or smaller than the corresponding signature of the plastics ensures that the method used to determine the type and presence of the plastics is able to separate the plastics from the mounting medium. In this regard, it is noted that the existing analyzing systems and their associated methods use a binding substance that has the density lower than the minerals of interest so that the signature of the binding substance can be filtered out during the processing stage. In this embodiment, the opposite is true, i.e., the binding substance (the mounting medium) has a density larger than the plastic of interest.

For example, the mounting medium may be composed of, but not is limited to, calcium sulfate hemihydrate or zinc phosphate hydrate, or a combination of these materials. In one embodiment, the mounting medium includes a combination of elements, compounds or materials. The mounting medium needs to be a water-insoluble solid at ambient temperature and may include the polymeric compounds of sulphur or sulphur-nitrogen or sulphur-carbon compounds; silicon compounds, phosphorous compounds, hydrates or anhydrates of clays, plasters and/or cements including glass-ionomer cements, hydraulic or non-hydrolic cements consisting of any mixture of, but not limited to; calcium silicate; calcium aluminate; calcium sulfoaluminate, calcium sulphate; alkali metal silicates; calcium aluminosilicate; magnesium oxychloride; magnesium hydroxide; zinc oxychloride; calcium hydroxide; zinc oxide eugenol; zinc oxide; zinc phosphate; zinc polycarboxylate; zinc polyacrylate; zinc methacrylate; zinc silicophosphate; zinc-/copper-substituted dicalcium silicate/ethoxy benzoic acid cements/orthoethoxybenzoic based cements. Other cations that may be used in the mounting medium in combination with, or in place of those in the above noted compounds, with the presence of the appropriate charge balance, may include ammonium, hydronium, nitronium, and/or cations of: aluminum; antimony, arsenic; bismuth, beryllium, barium; boron; calcium; chromium; copper; cesium; cobalt; cadmium; gold; gallium; iron; indium; iridium; lead; lithium; magnesium; molybdenum; manganese; mercury; nickel; osmium; potassium; palladium; platinum; rubidium; rhodium; ruthenium; rhenium; silver; scandium; sodium; strontium; tin; tungsten; titanium; tantalum; tellurium; thallium; vanadium; zinc; zirconium.

In one application, the anions of the above discussed compounds may be used in combination with, or replaced by, with the appropriate charge balance, the following anions, which may include any one or multiples of: antimonide; arsenate; arsenide; azide; acetate; ammonium nitrate; acetylide, azide; boride; bromide; bromate; bromite; bicarbonate; bisulphate; borohydride; borate; carbide; chloride; carbonate; chromate; chlorate; cyanide; cyanate; citrate; cyanamide; carbonyl; chlorite; dihydrogen phosphate; dithionite; fluoride; ferrate; ferrite; ferrioxalate; ferricyanide; ferrocyanide; fulminate; fluorosilicate; formate; hydroxide; hexachloroplatinate; hydride; hydrogen sulfate; hexafluorosilicate; hypochlorite; hypobromite; hydrogen fluoride; heptafluorotantalate; hexafluorophosphate; hydrosulfide; hydrogen phosphate; hydrogen sulfite; hydrogen carbonate; hydrogen bicarbonate; iodide; iodate; manganate; molybdate; monofluorophosphate; nitride; nitrate; nitrite nitroferricyanide; oxide; orthomolybdate; oxyfluoride; oxychloride; oxybromide; oxalate; phosphide; potassium sulphate; perchlorate; permanganate; persulfate; phosphate; peroxide; perbromate; periodate; pyrophosphate; silicate; sulfide; sulphate; sulfamate; silicide; selenate; selenide; selenite; sulfite; selenocyanate; telluride; titanate; tetrachloroaluminate; thiocyanate; triflate; tartrate; tetrachloroaluminate; tetrafluoroborate; thiosulfate; tungstate; vanadate; zirconate.

However, no matter which compound or combination of compounds is utilized in the inorganic mounting medium 402, it needs to have a signature different than the studied plastics.

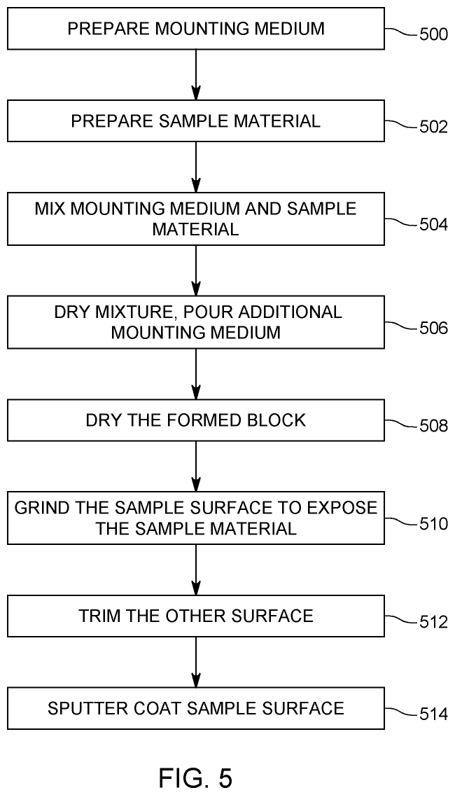

Next, the method illustrated in FIG. 3 includes a step 304 of preparing the probe/block 400A, or 400B or 400C for the QEMSCAN analyzing system, by mixing the sample plastic 410 and/or 412 with the inorganic mounting medium 402. Details of this step are now discussed with regard to FIG. 5. The sample preparation method illustrated in FIG. 5 includes a step 500 of preparing the mounting medium 402 by treating precursor materials to produce a paste. This may involve, for example, mixing the precursor material with pure water or a weak acid solution in a mass ratio ranging from 10 parts mounting medium and 15 parts liquid, to 20 parts mounting medium and 5 parts liquid.

Next, the sample material 406 is prepared in step 502. A quantity of the sample material 406, which includes the plastics 410 and/or 412 and the other material 404, is weighed and placed into a cylindrical plastic mold having a diameter between 10-50 mm, or an alternative enclosed cell, up to an approximate size of 100×65 mm. The sample material 406 may comprise up to 70% of the total mass of the block.

A quantity of the mounting medium paste is poured in step 504 into the mold to fill it to between approximately ¼ and ⅓, in order that the sample is contained in the outermost layer of the mounting medium. The mounting medium is then mixed with the sample material by stirring, for example, in a figure of eight motion, and simultaneously rotating the mold to ensure the sample material is completely covered by the mounting medium. The obtained mixed layer has a thickness of about 2-5 mm. To even out the mixture and ensure any air bubbles escape, the mold can be tapped gently.

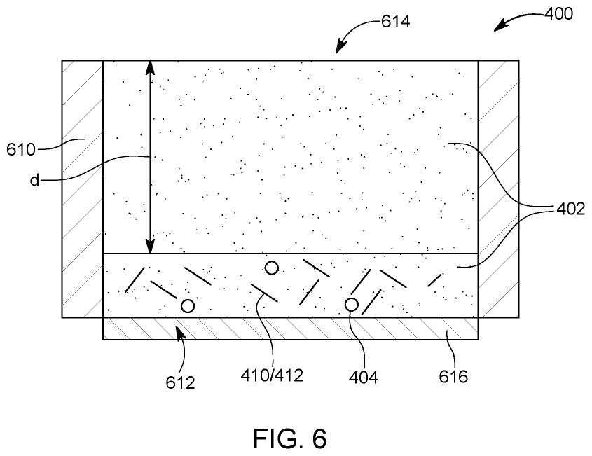

The above mixture is allowed in step 506 to dry for a couple minutes, and then further mounting medium 402 is poured into the mold 610 (without mixing) to encase the sample particles (i.e., plastics 410/412 and any other material 404 that may be present in the sample material 406) and ensure they are completely covered to a depth of up to 50 mm, as illustrated in FIG. 6. The probe/block 400 is left in step 508 to dry completely and solidify, either by air-drying at ambient temperature for several hours or overnight, and/or by oven-assisted drying, either with or without using pressure vessel compaction and/or vacuum chamber evacuation. Oven-assisted drying must be carried out at a temperature lower than that which may destroy, decompose or modify the plastic sample, surrounding material and/or the mounting medium. An ideal temperature is 40-50° C. and the block may be placed in the oven until completely dry, which may take 1-2 hours, depending on the mounting medium selected. Once completely dry and solid, the block is released from the mold.

The bottom face 612 of the block 400 is then ground down in step 510 using a grinding/polishing machine, equipped with, for example, a 220 MD plate followed by a 1200 MD plate. The block 400 is ground, polished, ion milled or processed by any other appropriate method until the sample material 406's surface is flat with the surrounding mounting medium 402 and until the block's surface is flat and smooth (see FIG. 3). The block 400 is then cleaned with water and/or a solvent such as ethanol after each step and dried with paper towel, lint free and/or microfiber cloth. The solvent selected must have no known solubilizing or modifying effect on the plastics and/or sediment within the sample or the mounting medium. In one application, this analysis may be used to analyze tyre particles, as these are largely rubber based synthetic polymer materials. The block 400 is allowed to dry again, either at ambient temperature or in an oven at a low heat (<50° C.). The back surface 614 of the block 400 is trimmed off in step 512, with a diamond saw or ground down, if required, to give a flat base and the block is then cleaned with a high-pressure air gun or non-contaminating aerosol air spray to remove any contaminant particles.

The block's surface 612, which exposes the plastic 410/412 of the sample material 406, is then sputter coated in step 514 to improve the signal-to-noise ratio during SEM analysis, to reduce charge build-up and prevent sample damage. A non-carbon coating material 616 is used, such as, but not limited to copper, gold, palladium, platinum, silver, chromium, iridium, tungsten or a combination of these. The coating material 616 may be formed to have a thickness between 1 and 200 nm.

A specific block 400 that is made according to the method discussed in FIG. 5 is now discussed. In one application, the mounting medium 402 is selected to be calcium sulfate hemihydrate (known as Crystacal). 100 g of Crystacal powder is mixed with 35 g of deionised water and the mixture is stirred into a smooth paste. Alternatively, in another application, the mounting medium 402 is selected to be calcium sulfate hemihydrate (known as Crystacast). 100 g of Crystacast powder is mixed with 29 g of deionized water and the mixture is stirred into a smooth paste.

In a third application, the mounting medium 402 is selected to be zinc phosphate cement. A zinc phosphate cement kit, such as tgZincem may be used, and this kit includes a zinc oxide-based powder mix and a phosphoric acid-containing liquid. Then, 2 cap measures of the powder (approx. 560 mg) and 12 drops of the liquid (approx. 715 mg) are placed on a pre-cooled glass slab (or 1.3-1.4 g powder to 0.5 ml liquid). The powder is divided into quarters, one quarter divided into two-eights, one eighth divided into two-sixteenths. The zinc oxide powder mix is mixed into the phosphoric acid-containing liquid in the order: 1/16, 1/16, ⅛, ¼, ¼, ¼. In this way, three different mounting materials 402 are obtained. Then, the other steps of the method illustrated in FIG. 5 are performed for forming the block 400.

To overcome some of the problems noted in the Background section (i.e., the epoxy resin and plastic material have the same chemistry and density and thus it is difficult to separate the two with the existing automatic mineralogic analyzing systems), researchers have tried mounting the samples in carnauba wax and supporting the sample in a ring of epoxy resin [1]. The wax has a lower average atomic number, enabling better contrast against the coal. However, it has a similar average atomic number to hydrogen-rich kerogen and thus, it is not versatile in imaging various carbon-based samples. In addition, because the wax is soft (hardness below 2 on Mohs scale), it may be smeared and contaminate the sample surface during polishing. In a different approach, halogenated epoxy resins have been used to image coal and carbon samples. Through the addition of 15 wt % iodoform to epoxy resin, researchers have successfully studied organic carbon in metalliferous black shales by automated SEM-based imaging. However, the authors comment that it is still difficult to detect organic carbon where it is very finely disseminated in bands less than 1 μm in thickness, causing systematic underestimating of the organic carbon content [2].

It is noted that in a publication from 2006, ‘plastic’ matter was listed as a phase identified by QEMSCAN analysis of a dust sample captured on plastic tape [3]. However, this approach is limited because it would not be able to detect or quantify plastic within a larger volume of solid surrounding material. Furthermore, the tape as a mounting medium is sub-optimal because it would not allow the grinding/polishing to a flat surface, which is required for accurate EDS identification of a heterogeneous and densely populated sample (e.g., sediment) when using the existing analyzing systems.

Once the probe/block 400 is obtained as discussed above, it can be used in the existing analyzing systems, for example, QEMSCAN. In one embodiment, the hardness of the inorganic mounting medium 402 is selected to be at least 2, or at least 3 on the Mohs scale, so that the surface of the probe 400 can be mechanically treated for the purposes of QEMSCAN analysis. The QEMSCAN automated analysis system includes a SEM, which is reprogrammed to include the identification of particles with low BSE brightness, such as plastic. The difference in densities and chemical compositions of the mounting medium 402 and plastic 410/412 from the sample material 406 enables the visualization and characterization of the plastic. The area value for the surrounding mounting medium (external from the particles) is removed from the total so that the plastic and other material/minerals is normalized to 100%. Void or pore space within (internal to) the sample is quantified by calculation from the % area of the mounting medium contained within the boundaries of the particles. The results are then subjected to standard QEMSCAN processing tools.

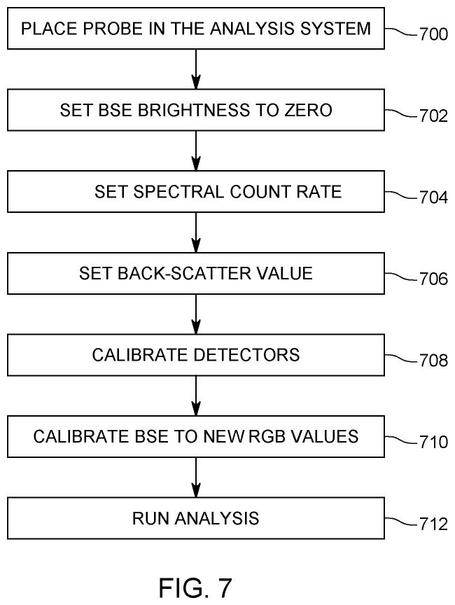

The procedure used for QEMSCAN analysis is now discussed in more detail with regard to FIG. 7. The QEMSCAN has various software modules, i.e., iExplorer, iMeasure, etc., which are known in the art and thus, their description is omitted herein. Although the other existing automatic mineralogic analysis systems have different software modules, the steps discussed herein are applicable to each of them. In step 700, the block/probe 400 prepared as discussed above, with an inorganic mounting medium 402, is placed in the analyzing system for analysis. The existing automatic mineralogic systems set the cut off BSE brightness to 25 so that the epoxy fixing medium is ignored by the system. However, contrary to the traditional methods, the current method sets in step 702 the BSE brightness at substantially zero, to capture those materials that have a low density, i.e., organic materials that include the plastic. This change may be implemented in the used automatic mineralogic analysis system by the user. Because of this fundamental change made to the methodology of the automatic mineralogic analysis system, the presence of the plastic that has a density below 25 RBG, in the block 400, may be detected. Based on the determined density of the plastic, the BSE brightness may be adjusted upward, for example, to be about 5, if all the plastic has a BSE brightness above this value. However, the BSE brightness in the current method is set up to a smaller value than the traditional BSE brightness of 25 that is used by the existing automatic mineralogic analysis system, and in this context, the term “substantially zero” is understood herein to mean any value smaller than 25. The aim for this step is to capture the plastic material and not to filter it out, as the existing systems are doing.

In addition, the spectral count rate is set in step 704 to be between 100and 5000 cps (counts per (pixel) spectrum, i.e., each point analyzed (pixel) will acquire an elemental spectrum of no less than 1,000 x-ray counts per pixel spectrum (between 100 and 5000 in another embodiment), but this measurement rate occurs many times per second, e.g., ˜200-300 times per second). This value represents how many measurements are taken per second for the energy emitted by the probe, as a consequence of the excitation of the probe's atoms by the incoming x-rays or other beams used for analysis. This value is different than what the traditional automatic mineralogic analysis systems are doing for hydrocarbon and mining industries, which have this value in the range of 100 kcps (kilo counts per second), for the following reasons. To achieve an acquisition rate of 1,000 counts per pixel spectrum analyzing around 200-300 pixels per second, generally requires an electron input of >200 kcps. QEMSCAN typically runs at an input rate of 400 kcps, and allowing for deadtime, i.e., 400 kcps allows a delivery of 200 kcps with 50% deadtime, where the deadtime means that the processing power is balanced between signal detection and data processing, this leads to the rate of ˜200-300 pixels per second. For the method discussed herein, the input rate is slowed down to 90 kcps. This means that in this embodiment the method is taking longer to obtain the same 1,000 cps (counts per pixel spectrum) acquisition and this helps to obtain a more favorable carbon spectral peak. This is so because the operator of this method is particularly interested in carbon (low density), whereas for normal mineralogical analysis, the operator is interested in many other elements with higher densities. Higher density elements/minerals produce fewer x-rays with the same energy, hence it is better to increase the throughput and so increase the number of x-rays from these elements. Thus, the traditional QEMSCAN method is optimized across the periodic table while in this method the optimization performance is reduced in favor of a simpler analysis.

In other words, this step slows down the data acquiring process as the system dwells longer at each spot for collecting the emitted energy.

Next, the iMeasure module's settings are reconfigured in step 706 to disable the back-scatter cut-off, i.e., set the back-scatter cut-off to 0. This means that the software is configured to measure the full range of RGB 0-255 and not ignore anything. X-rays and density data will be recorded for every pixel regardless of what is present there. Further, the iMeasure module's rate of electron energy disperse from the electron emitter is also modified to set to between 90 and 60 kcps (note that this is reduced from 400 kcps, which is the typical value for mineral detection) to better define the carbon peak for optimal carbon ‘speciation’. In step 708, the EDS detectors are re-calibrated to the selected rate of 90 kcps, using a corresponding EDS software (Bruker ESpirit for QEMSCAN) using a pure sample of Cu as a calibration method.

In step 710, the BSE is calibrated to new RGB values, for example, Cu is assigned a value of about 230, and Si is assigned a value of about 76, where the term “about” is understood in this context to mean plus or minus 30% of the noted value. Note that the traditional automatic mineralogic analysis systems assign different intensity values to these elements, for example, Au, which is very dense, is assigned the value 232, and Cu, which is not so dense, is assigned the value 130. Those skilled in the art should understand that these values can differ from system to system, but the principle remains the same, i.e., the traditional automatic mineralogic analysis systems assign a high value to one of the most dense element (Au) that is likely to be found in the sample probe to be analyzed, while the present method, in a sense, cuts off the dense materials at 255. This means that the method assigns to Cu, which is not so dense, the high value of 230 (255 is the maximum intensity value). As a result of this step, the gold would be only slightly brighter than copper, but looking at the chemical data, it is possible to distinguish one bright spot as gold, and another almost identical bright spot as copper. Therefore, on the intensity spectrum collected by the system, the high-density materials are squeezed in the range 230-255, while the low-density materials, that include the plastic, are stretched over the range 0-230. This would ensure that the system is highly sensitive to the plastic, i.e., the low-density materials. In step 712, the analysis of the sample material is run with an accelerating voltage in a range of 5-15 keV, and a sample current between 1-11 nA. Those skilled in the art would understand that these values may change from system to system, depending on the characteristics of the system.

The analysis step 712 may include plural sub-steps, one of which is the assignment of regions of the probe 400 as either plastic or organic or non-plastic carbon-based or natural matter (such as coal, wood, etc. that may be present in the surrounding material) using, for example, an iDiscover Species Identification Protocol (SIP) that is specific for QEMSCAN. Other modules need to be used for the other automatic mineralogic analysis systems. Step 712 involves the compilation of a database of EDS spectra from plastic and non-plastic carbon-based matter, captured by quantitative EDS analysis using line, spot and element mapping measurements from defined standards using the same SEM as used for sample analysis and the sample SEM setup parameters discussed above (e.g., 90 kcps, etc.). Standards may be prepared using the same mounting medium as the samples under analysis in order to obtain characteristic EDS spectra for the dictionary, or as standalone standards mounted on traditional SEM stubs. Once captured and added to the SIP dictionary, additional variations are made of these standard measurements, including plastic-plastic, plastic-mineral, mineral-mineral, plastic-organic, etc., boundary phases. This accounts for pixel measurements where two or more different types of particles and mineral are present, but they are smaller than the scanning resolution (e.g., 10-micron particles within a 50-micron pixel size). The dictionary can then recognize what a mix of particular things would look like from a chemical point of view. In the same vein, once enough standards are in the dictionary, additional theoretical and statistical variations can be added. This database enables the discrimination of features within the sample based on their EDS spectral data.

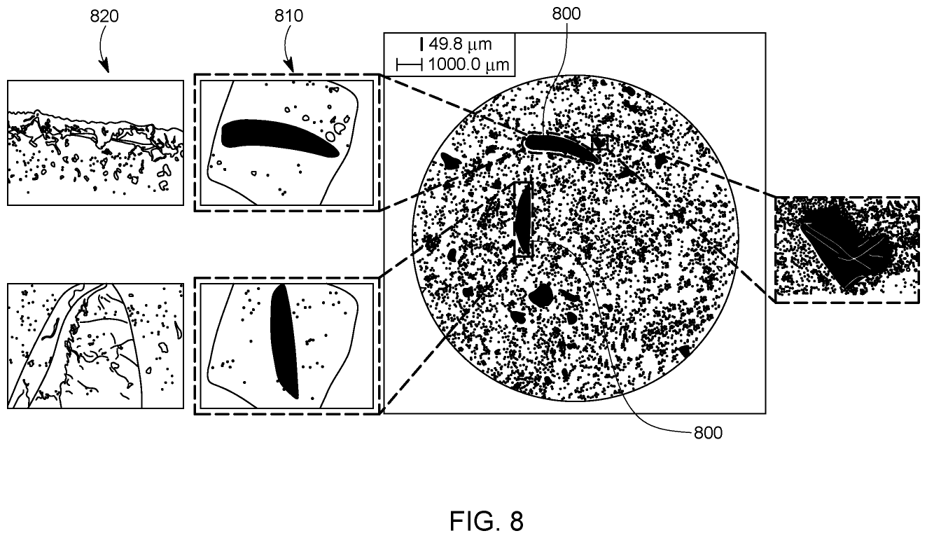

The final data output includes, but is not limited to, plastic content, physical and textural characteristics of particles, angularity and particle size distribution. The data can be displayed as a mineral map image with different materials highlighted in different colors, as shown in FIG. 8, and the physical characteristics can be grouped according to various parameters. Once key samples of interest have been identified, manual SEM analysis can reveal important insights into plastic structures, textural properties, degeneration and chemical alteration. FIG. 8 shows plastic particles 800 and corresponding SEM images 810 and 820 at different scales, e.g., millimeter scale for image 810 and micrometer scale for image 820.

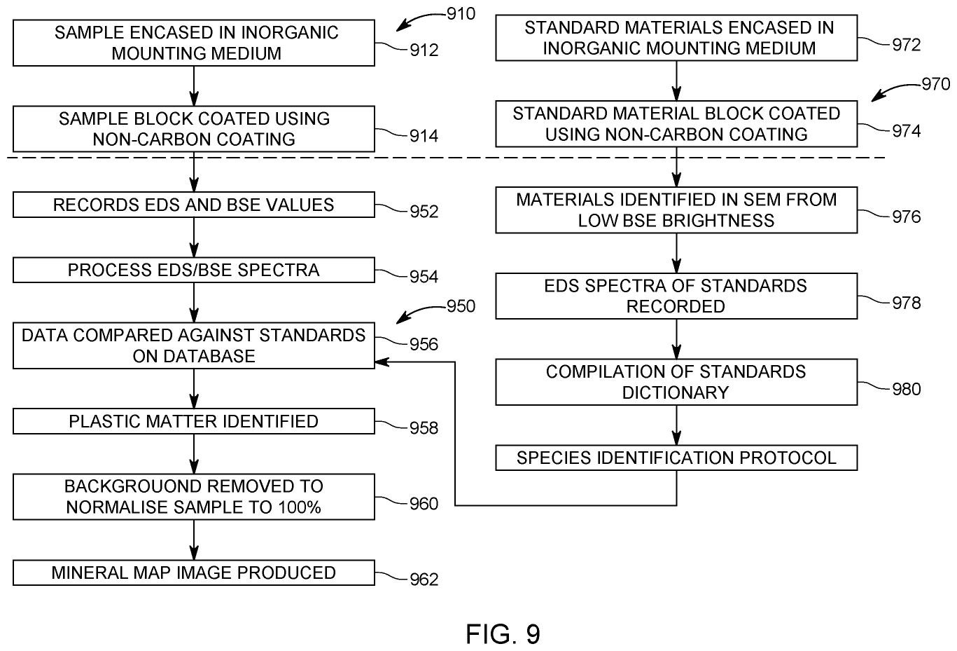

A comprehensive flow diagram for all phases of the methodology discussed in the above embodiments is illustrated in FIG. 9. The flow diagram includes a sample preparation part 910, an analysis part 950, and a building of the standards dictionary part 970. The sample preparation part 910 includes a step 912 of encasing the carbon sample in an inorganic mounting medium, as previously discussed above with regard to FIG. 5. In step 914, the sample block is optionally coated with the non-carbon coating layer 616 illustrated in FIG. 6.

The building of the standards dictionary part 970 includes a step 972 of encapsulating a known, standard material, in the inorganic mounting medium or mounting standalone on a standard SEM stub and a step 974 for coating the obtained block with the non-carbon coating layer 616. This block, whose chemical composition is known precisely, is used to create the dictionary of materials in step 976, by identifying the know materials in SEM from low BSE brightness. The EDS spectra of standards is recorded in step 978 and a compilation of the entire dictionary is performed in step 980, based on the plural known materials, which are prepared in steps 972 and 974. Steps 972 and 974 are similar to steps 912 and 914 with the exception that the sample material is known in steps 972 and 974 and unknown in steps 912 and 914.

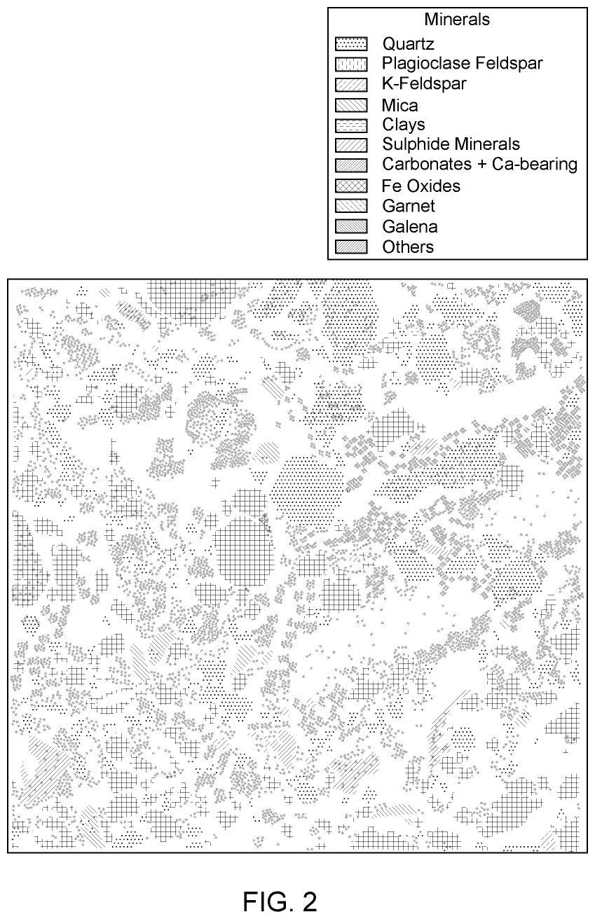

The analysis part 950 receives in step 952 the measured energies of the coated block from step 914, and records the EDS and BSE values, then, in step 954, the method processes the EDS and BSE spectra and extracts the peaks of these spectra, and in step 956 the extracted peaks are compared against the standards generated in part 970, to identify in step 958 the plastic materials. The analysis part 950 may also include a step 960 of removing the medium (e.g., Crystacal) from the measured spectra to normalize the sample to 100%, and a step 962 of generating the mineral map image, like in FIG. 2, which may be kept in electronic form, displayed on a screen, or printed in paper.

One additional step may be added to the method, for example, to provide a number of different software processing tools that can be used when finalizing a sample dataset. These are provided with the manufacturer software. There are a range of these that might be applied, but one used in particular would be the “boundary phase processor.” Once the dictionary has distinguished different particles, and also classified some pixels that themselves represent a boundary phase (e.g., 75% plastic and 25% quartz, or 25% plastic and 75% quartz), the boundary phase processor can be used as a final clean-up where all pixels are compared to surrounding pixels, and any unclassified pixels can be statistically classified. For example, this step may be applied to a single unclassified pixel within a plastic particle, which does not match any entry in the dictionary. By checking the surrounding pixels, the processor would assign this as plastic. Whereas the dictionary may have many mineral-plastic definitions based on e.g., 75:25, 85:15, etc, as well as all the entries for standards, theoretical standards, and modified theoretical standards, to account for all mixed-pixel possibilities and cross-reference all entries in the dictionary would create a massively long and impractical list. The boundary phase processor therefore acts as a final statistical clean-up. If an unclassified pixel is surrounded by more unclassified pixels, these would be left as unclassified.

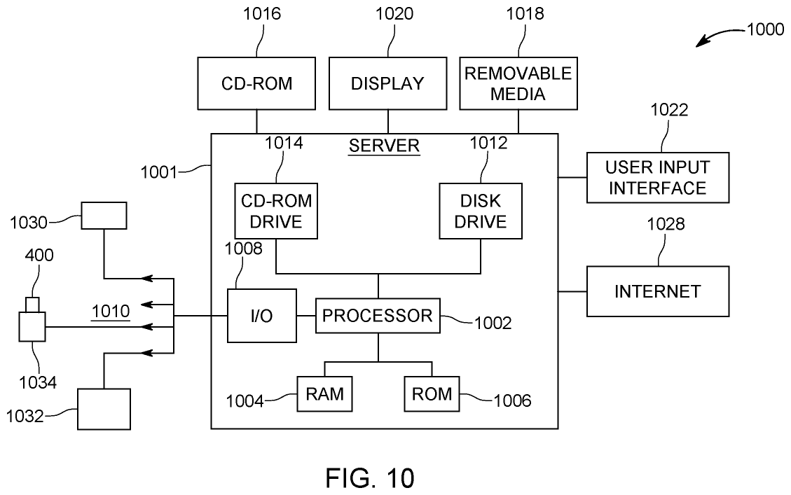

The above-discussed procedures and methods may be implemented in the processor of the automated mineralogic analyzing system as illustrated in FIG. 10. Hardware, firmware, software or a combination thereof may be used to perform the various steps and operations described herein. Analyzing system 1000 of FIG. 10 is an exemplary computing structure that may be used in connection with such a system.

Mineralogic analyzing system 1000 suitable for performing the activities described in the exemplary embodiments may include a server 1001. Such a server 1001 may include a central processor (CPU) 1002 coupled to a random access memory (RAM) 1004 and to a read-only memory (ROM) 1006. ROM 1006 may also be other types of storage media to store programs, such as programmable ROM (PROM), erasable PROM (EPROM), etc. Processor 1002 may communicate with other internal and external components through input/output (I/O) circuitry 1008 and bussing 1010 to provide control signals and the like. Processor 1002 carries out a variety of functions as are known in the art, as dictated by software and/or firmware instructions.

Server 1001 may also include one or more data storage devices, including hard drives 1012, CD-ROM drives 1014 and other hardware capable of reading and/or storing information, such as DVD, etc. In one embodiment, software for carrying out the above-discussed steps may be stored and distributed on a CD-ROM or DVD 1016, a USB storage device 1018 or other form of media capable of portably storing information. These storage media may be inserted into, and read by, devices such as CD-ROM drive 1014, disk drive 1012, etc. Server 1001 may be coupled to a display 1020, which may be any type of known display or presentation screen, such as LCD, plasma display, cathode ray tube (CRT), etc. A user input interface 1022 is provided, including one or more user interface mechanisms such as a mouse, keyboard, microphone, touchpad, touch screen, voice-recognition system, etc.

Server 1001 may be coupled to other devices, such as electromagnetic sources (e.g., X-ray), SEM, etc., 1030, energy detectors 1032, a support assembly 1034 that holds the block 400, etc. The server may be part of a larger network configuration as in a global area network (GAN) such as the Internet 1028, which allows ultimate connection to various landline and/or mobile computing devices.

The disclosed embodiments provide a method for preparing an organic sample with an inorganic mounting medium and a method for analyzing such a sample with an automatic mineralogic analyzing system. It should be understood that this description is not intended to limit the invention. On the contrary, the embodiments are intended to cover alternatives, modifications and equivalents, which are included in the spirit and scope of the invention as defined by the appended claims. Further, in the detailed description of the embodiments, numerous specific details are set forth in order to provide a comprehensive understanding of the claimed invention. However, one skilled in the art would understand that various embodiments may be practiced without such specific details.

Although the features and elements of the present embodiments are described in the embodiments in particular combinations, each feature or element can be used alone without the other features and elements of the embodiments or in various combinations with or without other features and elements disclosed herein.

This written description uses examples of the subject matter disclosed to enable any person skilled in the art to practice the same, including making and using any devices or systems and performing any incorporated methods. The patentable scope of the subject matter is defined by the claims, and may include other examples that occur to those skilled in the art. Such other examples are intended to be within the scope of the claims.

### REFERENCES

The entire content of all the publications listed herein is incorporated by reference in this patent application.
[1] G. O'Brien, Y. Gu, B. J. I. Adair, B. Firth, “The use of optical reflected light and SEM imaging systems to provide quantitative coal characterisation,” Minerals Engineering, 2011, 24, 1299-1304.
[2] Anne Rahfeld, Jens Gutzmer, “MLA-Based Detection of Organic Matter with lodized Epoxy Resin—An Alternative to Carnauba,” Journal of Minerals and Materials Characterization and Engineering, 2017, 5, 198-208.
[3] I. Tonzetic, A. Butcher, A. F. Cropp, C. Pudmenzky, “Automated SEM analysis (measurement & characterisation) of dust using QEMSCAN, 2006, https://www.researchgate.net/publication/276323878_Automated_SEM_Analysis_Mea surement_Characterisation_of_Dust_Using_QEMSCAN.

1. A method for preparing a block for chemical analysis, the method comprising:
providing a substantially inorganic mounting medium;
providing a sample material that includes plastic and another material;
mixing the substantially inorganic mounting medium with the sample material to generate the block; and
smoothing a first surface of the block to expose the plastic.

2. The method of claim 1, wherein the step of mixing further comprises:
placing the substantially inorganic mounting medium and the sample material in a mold; and
adding additional substantially inorganic mounting medium, in the mold, over the mixed substantially inorganic mounting medium and the sample material to form the block.

3. The method of claim 2, further comprising:
drying the formed block; and
processing a surface of the block that includes the sample material to expose the sample material and to smooth the surface.

4. The method of claim 1, further comprising:
sputter coating a surface of the block, which exposes the sample material, with a non-carbon material, to cover the sample material,
where the non-carbon material is free of carbon.

5. The method of claim 1, wherein the substantially inorganic mounting medium includes less than 20% by mass organic material.

6. The method of claim 1, wherein the substantially inorganic mounting medium includes only inorganic materials.

7. A block for chemical analysis in an automatic mineralogic analysis system, the block comprising:
a substantially inorganic mounting medium;
a sample material that includes plastic and another material;
a first face of the block includes a part of each of the substantially inorganic mounting material, the plastic and the another material; and
a second surface, opposite to the first surface and including only the substantially inorganic mounting medium.

8. The block of claim 7, further comprising:
a coating layer formed on the first surface of the block, wherein the coating layer is free of carbon.

9. The block of claim 7, wherein the substantially inorganic mounting medium includes less than 20% by mass organic material.

10. The block of claim 7, wherein the substantially inorganic mounting medium includes only inorganic materials.

11. The block of claim 7, wherein the substantially inorganic mounting medium includes a metal or metalloid directly bonded to a carbon atom.

12. A method for analyzing a probe with a mineralogic analyzing system, the method comprising:
placing the probe in the mineralogic analyzing system;
setting a backscattered electron (BSE) collection brightness at substantially zero to capture carbon-based materials;
setting a spectral count rate between 100 and 5000 counts per pixel spectrum for capturing the carbon-based materials; and
performing an analysis of the probe to identify a plastic present in the probe,
wherein the probe includes the plastic and a substantially inorganic mounting medium.

13. The method of claim 12, further comprising:
calibrating the BSE collection to new red, green and blue values so that a copper atom corresponds to a value of about 230.

14. The method of claim 13, further comprising:
calibrating the BSE collection so that a silicon atom corresponds to a value of about 76.

15. The method of claim 12, further comprising:
building a dictionary of minerals and elements using exclusively the substantially inorganic mounting medium.

16. The method of claim 15, further comprising:
self-referencing the dictionary of minerals and elements for new unknown organic and synthetic polymers.

17. The method of claim 12, further comprising:
sputter coating a surface of the block, which exposes the sample material, with a non-carbon material, to cover the sample material,
wherein the non-carbon material is free of carbon.

18. The method of claim 13, wherein the substantially inorganic mounting medium includes less than 20% by mass organic material.

19. The method of claim 13, wherein the substantially inorganic mounting medium includes only inorganic materials.

20. A non-transitory computer readable medium including computer executable instructions, wherein the instructions, when executed by a processor, implement a method for analyzing a probe with a mineralogic analyzing system, the method comprising:
receiving the probe in the mineralogic analyzing system;
setting a backscattered electron (BSE) collection brightness at substantially zero to capture carbon-based materials;
setting a spectral count rate between 100 and 3000 counts per pixel spectrum or less for capturing the carbon-based materials; and
performing an analysis of the probe to identify a plastic present in the probe,
wherein the probe includes the plastic and a substantially inorganic mounting medium.
